(19) United States
(12) Patent Application Publication
(10) Pub. No.: US 2022/0108765 A1
(43) Pub. Date: Apr. 7, 2022

(54) SAMPLING UNIQUE MOLECULAR STRUCTURES FROM AUTOENCODERS

(71) Applicant: International Business Machines Corporation, Armonk, NY (US)

(72) Inventors: TAKESHI TESHIMA, Tokyo (JP); Hiroshi Kajino, Tokyo (JP)

(21) Appl. No.: 17/060,862

(22) Filed: Oct. 1, 2020

(51) Int. Cl. G16B 15/00; G06N 3/04; G06N 3/08; G06N 7/00

(52) U.S. Cl. CPC G16B 15/00; G06N 7/005; G06N 3/08; G06N 3/049

(57) ABSTRACT

A system, method, and computer program product for computational molecular design are disclosed. The method includes receiving an input molecule, encoding the input molecule as a vector in latent space, identifying a target region in the latent pace, sampling latent vectors from the target region, and generating two or more discrete representations of molecules for each of the sampled latent vectors by decoding the sampled latent vectors via sequential decision-making, which includes selecting most likely symbols at each step. Further, the method includes outputting, for each sampled latent vector, a unique molecule selected from the discrete representations of molecules. The system includes at least one processing component, at least one memory component, an encoder, a sampling module, and a decoder, which are configured to carry out the method. The computer program product includes a computer readable storage medium having program instructions to cause a device to perform the method.

### BACKGROUND

The present disclosure relates to computational molecular design with variational autoencoders (VAEs) and, more specifically, to sampling of unique molecules using beam search.

Computational molecular design is used to generate chemical structures most likely to fit a set of desired parameters. For example, VAEs can encode molecular structures as vectors in continuous latent space. Generated latent vectors in the continuous space can be sampled by a decoder, which maps the latent vectors to discrete representations of novel molecules. Bayesian optimization techniques can be used to sample novel molecules predicted to maximize a given objective (e.g., new drug candidates).

### SUMMARY

Various embodiments are directed to a system that includes at least one processing component, at least one memory component, an encoder, a sampling module, and a decoder. The encoder and decoder can be based on a recurrent neural network. The encoder is configured to receive an input molecule, and encode the input molecule as a vector in latent space. The sampling module is configured to identify a target region in the latent space. The sampling module can identify the target region based on a probability distribution on the latent space. The decoder is configured to sample latent vectors from the target region, and generate discrete representations of molecules for the sampled latent vectors by decoding the sampled latent vectors via sequential decision-making with a beam search module configured to select a number of most likely symbols at each step in the sequential decision-making. The number of most likely symbols can be greater than 1, and can include symbols having the highest probabilities of satisfying a given constraint (e.g., a quantitative estimate of drug likeness value). The decoder is also configured to output, for each of the sampled latent vectors, a unique molecule selected from the discrete representations. The system can also include a predictor configured to predict molecular properties based on latent space representations of molecules and/or a predictor configured to predict molecular properties based on discrete representations of molecules.

Further embodiments are directed to a method of computational molecular design, which includes receiving an input molecule, encoding the input molecule as a vector in latent space, identifying a target region in the latent pace, and sampling latent vectors from the target region. The target region can be identified based on a probability distribution on the latent space. The method also includes generating two or more discrete representations of molecules for each of the sampled latent vectors by decoding the sampled latent vectors via sequential decision-making, which includes selecting a number of most likely symbols at each step. The number of most likely symbols can be greater than 1, and the most likely symbols can be those having the highest probabilities of satisfying a give constraint (e.g., a quantitative estimate of drug likeness value). Further, the method includes outputting, for each of the sampled latent vectors, a unique molecule selected from the two or more discrete representations of molecules. The method can also include predicting molecular properties based on latent space representations of molecules and/or discrete representations of molecules. The encoding and decoding can be carried out with a recurrent neural network.

Additional embodiments are directed to a computer program product for computational molecular design. The computer program product includes a computer readable storage medium having program instructions embodied therewith, the program instructions executable by a processor to cause a device to perform a method. The method includes receiving an input molecule, encoding the input molecule as a vector in latent space, identifying a target region in the latent pace, and sampling latent vectors from the target region. The target region can be identified based on a probability distribution on the latent space. The method also includes generating two or more discrete representations of molecules for each of the sampled latent vectors by decoding the sampled latent vectors via sequential decision-making, which includes selecting a number of most likely symbols at each step. The number of most likely symbols can be greater than 1, and the most likely symbols can be those having the highest probabilities of satisfying a give constraint. Further, the method includes outputting, for each of the sampled latent vectors, a unique molecule selected from the two or more discrete representations of molecules. The encoding and decoding can be carried out with a recurrent neural network.

### BRIEF DESCRIPTION OF THE DRAWINGS

FIG. 1 is a block diagram illustrating a molecular design environment, according to some embodiments of the present disclosure.

FIG. 2 is a flow diagram illustrating a process of molecular design, according to some embodiments of the present disclosure.

FIG. 3 is a block diagram illustrating a computer system, according to some embodiments of the present disclosure.

FIG. 4 is a block diagram illustrating a cloud computing environment, according to some embodiments of the present disclosure.

FIG. 5 is a block diagram illustrating a set of functional abstraction model layers provided by the cloud computing environment, according to some embodiments of the present disclosure.

### DETAILED DESCRIPTION

Molecular design includes techniques for designing new molecules based on a set of parameters. These parameters can include chemical and/or physical properties such as structural elements, solubility, electronegativity, toxicity, reactivity, size, polarity, synthetic complexity, etc. One of the largest applications of molecular design is in drug discovery. It is estimated that approximately 10⁸ drug candidate molecules have been synthesized. However, estimates of the number of potential drug candidates range from 10²³-10⁶⁰ different substances. This makes synthesis and testing of the full range of drug candidate molecules impractical, if not impossible.

Before the availability of computational techniques, molecular design was based entirely on human knowledge and experimentation. Computational molecular design systems can supplement this process by locating (e.g., in a database) or generating chemical structures most likely to fit a set of desired parameters or maximize a given objective. For example, a user can input a chemical structure in a machine-readable format (e.g., with line notation-based encoding such as simplified molecular-input line-entry system (SMILES) or a graph-based representation). A computational system can then locate similar molecular structures in a structure database, likely properties of the input molecule, etc. The computational system can also generate novel structures based on information input by a user. For example, the user can input a structure of a known compound and instructions to identify potentially useful analogues of this compound. Various techniques can then be used to carry out these instructions, such as searching large libraries of compounds and using genetic algorithms to perform local searches over discrete spaces. However, these techniques do not allow searches to be carried out over open-ended, continuous space.

Machine-learning processes enable more efficient searching over the vast molecular space. There are at least two applications. One is to use machine learning techniques to generate novel molecules for optimization of desired chemical and physical properties. Potentially useful generated compounds can be identified using Bayesian optimization techniques. For example, variational autoencoders (VAEs) can encode representations of molecular structures as vectors in continuous latent space. Bayesian optimization or gradient-based optimization can then be used to select structures in the latent space that maximize a given objective. Another application is to generate a large number of novel molecules that satisfy certain constraints on chemical/physical properties. VAEs are also used to obtain continuous representations of molecular structures, but sampling techniques (e.g., approximate Bayesian computation (ABC), sequential Monte Carlo (SMC), etc.) can be combined so as to efficiently sample molecules that satisfy the constraints.

The sampling application has limited applicability due to its low sampling efficiency. In the sampling algorithms, first, continuous representations are sampled, and then they are decoded via the decoder of VAE. However, when these structures are decoded, a large number of non-unique structures are often output, thereby reducing the effective sample size. This can occur when the VAE decoder maps more than one latent vector to the same molecule in discrete molecular space.

Techniques for increasing the effective sample size of novel candidate molecules by reducing the number of output non-unique molecules are disclosed herein. When a molecular structure is input in a machine-readable format, an autoencoder converts the molecular structure into a vector in continuous latent space. The autoencoder decodes new molecules generated in the latent space using sampling techniques to sample latent vectors based on given parameters (e.g., Log P<1.0 (P=partition coefficient), MolWeight <150, etc.). Additional examples of parameters and objectives are discussed in greater detail below. The beam search can then be employed in decoding the sampled latent vectors via sequential decision-making processes. This reduces the number of non-unique molecules output when the sampled latent vectors are mapped to discrete representations.

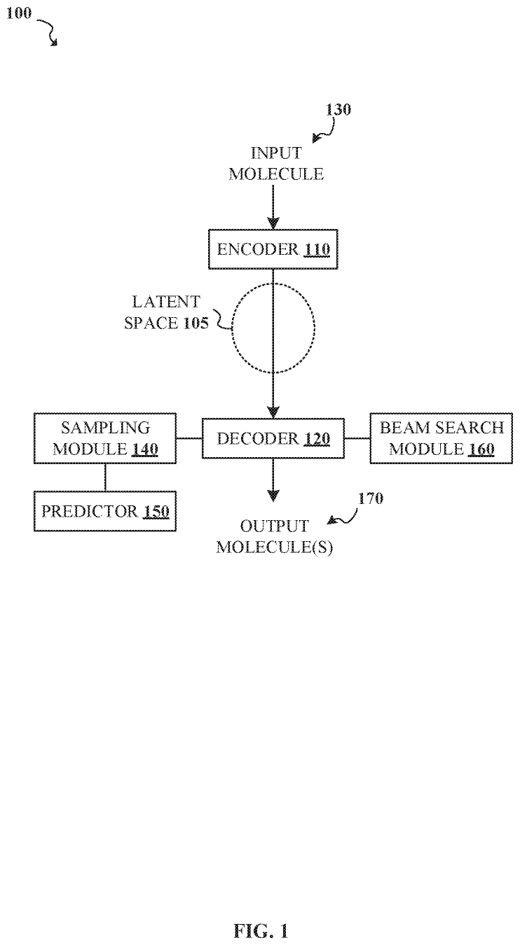

FIG. 1 is a block diagram illustrating a molecular design environment 100, according to some embodiments of the present disclosure. The molecular design environment 100 uses an autoencoder, such as a variational autoencoder (VAE), to map molecular structures to and from latent space 105. The autoencoder includes an encoder 110 and a decoder 120. The molecular design environment 100 also includes an input molecule 130, a sampling module 140, a predictor 150, a beam search module 160, and at least one output molecule 170.

The input molecule 130 is a machine-readable representation of any molecule selected automatically or by a user. For example, the input molecule 130 can be a particular drug molecule, molecular catalyst, polymer resin, etc. Examples of machine-readable molecular representation formats can include line notations and molecular graphs. Examples of line notations can include SMILES and International Union of Pure and Applied Chemistry (IUPAC) chemical identifier (InChI) formats. SMILES can use character strings, lists of tokens, parse trees, and/or molecular graphs to represent valence bond models, and can specify features such as atoms, bonds, rings, branching, disconnections, isomerism, and reactivity. While molecules represented by SMILES character sequences are illustrated herein, any appropriate molecular representation can be used in some embodiments.

Both the encoder 110 and decoder 120 can be trained using variational learning. In some embodiments, this training uses a generative model trained on SMILES character sequences via a recurrent neural network (RNN) such as a long short-term memory (LSTM) network or gated recurrent unit (GRU). The RNN or, in some embodiments, another neural network (e.g., convolutional neural network (CNN)) can be used to associate molecular properties (e.g., solubility, toxicity, activity, solvation energy, polarity, synthetic accessibility (SA), photovoltaic efficiency, molecular weight, quantitative estimate of drug likeness (QED), etc.) with molecular structure representations. Examples of training data sources can include molecular structure databases (e.g., QM9 dataset, ZINC dataset, etc.), other chemical databases, molecular structures extracted from journal articles, patent filings, or other publicly available documents, etc.

The encoder 110 encodes the character sequence of the input molecule 130 into a vector in continuous latent space 105. Latent vectors representing new molecular structures can then be generated using latent space operations. The sampling module 140 can use sampling techniques (e.g., ABC) to specify a prior distribution over the latent space 105 to identify a target region in the latent space 105. The target region can be defined by a probability distribution on the latent space 105, where latent vectors sampled from the target region have the largest probabilities of satisfying a given constraint. However, in some embodiments, the target region can be a region of the latent space with a uniform distribution.

The probability distribution can be determined based on property predictions made by the predictor 150, which can be a machine-learning model trained to associate properties with latent vectors representing molecular structures or a simulator designed to predict properties based on discrete molecular structures. The target region corresponds to structures with the largest probabilities of satisfying one or more given constraints (e.g., a range of log P values, a minimum and/or maximum molecular weight, a threshold QED value, a threshold SA value, etc.).

The decoder 120 can sample a number (N) of latent vectors, {zₙ}ₙ₌₁^{N}˜p(z), from the target region in the latent space 105, based on a prior distribution (p(z)) specified by the sampling module 140. The decoder 120 converts each sampled latent vector z to an output molecule (M) 170, which can be represented by a set of symbols (Σ) where each symbol represents an element (m) of the output molecule 170. For example, the symbols can represent characters in a SMILES character sequence. The decoder 120 can use a sequential model (e.g., RNN, LSTM, GRU, etc.) of the symbols such as q(σₜ|σ_{1:t-1}, z)(σₜ∈Σ), where σ_{1:t}={σ₁, . . . σₜ}. The decoding process can be represented by equation 1:

Dec: σ_{1:t} [symbol] m∈ [symbol] (1)

The decoder 120 uses the beam search module 160 to select a number (K) of most likely symbols at each step in a sequential decision-making process, where K is the beam width used by the beam search module 160. This can be represented by equation 2:

$${\{ {σ}_{1 : {t}_{n}}^{( n ) , k} \}}_{k = 1}^{K} = BeamSearc h ( {z}_{n} , q , K ) \qquad ( 2 )$$

In the first step of the decoding process, the beam search module 160 selects a number (k) of symbols having the highest probabilities of being the first symbol (e.g., the first character in a SMILES character sequence), based on the input molecule 130. For example, if K=3, the three most likely first symbols can be selected in step 1. The probabilities used to determine the likelihood of being the first symbol are calculated based on the input molecule 130. In the second step, the beam search module 160 can select the top k pairs of first and second symbols. The selected pairs have the highest probabilities of being the first pair of symbols, based on the k most likely symbols selected at step 1. Similarly, in the third step, the beam search module 160 selects the k most likely first, second, and third symbols in the sequence based on the pairs selected in the previous step. The decoder 120 converts the top k complete sequences to molecular representations. This can be represented by equation 3:

$${\{ {m}^{( n ) , k} \}}_{k = 1}^{K} = Dec ( {\{ {σ}_{1 : {t}_{n}}^{( n ) , k} \}}_{k = 1}^{K} ) \qquad ( 3 )$$

This process can be repeated for each n=1, . . . , N sampled vector. A unique molecule is then selected from the decoded most likely molecules for each of the sampled vectors. This can be represented by equation 4:

$${k}^{⋆} = {min}_{1 \leq k \leq K , {m}^{( n ) , k} ∉ output} k \qquad ( 4 )$$

which results in:

output=output ∪{m^{(n),k}*} (5)

In some embodiments, the sampling module 140 places further constraints on the selection of output molecules 170. For example, the sampling module 140 can filter out invalid structures, such as SMILES strings that are not syntactically valid and semantically coherent. The sampling module 140 can also filter out structures that do not represent novel molecules (e.g., molecular structures present in the training data).

The decoder 120 outputs a subset of [symbol] _{K}={m^{(n),k}|n=1, . . . , N, k=1, . . . , K} (illustrated by output molecule(s) 170) such that the subset contains fewer non-unique molecules. That is, when beam search is used in the decoding of N sampled latent vectors, the output molecules (M_{K}) 170 include a number of unique molecules represented by #Uni(M_{K}). It can be shown that, given the same set of latent vectors {zₙ}ₙ₌₁^{N}, the use of beam search does not result in a decreased number of unique molecules as compared to the number of unique molecules (#Uni(M₁)) output when K=1 (i.e., without the use of beam search). This can be expressed as #Uni(M₁)≤#Uni(M₂)≤ . . . ≤#Uni(M_{K}). For example, if #Uni(M₁)=N, then #Uni(M_{K})≥N holds, and uniqueness of the output molecules 170 does not decrease with the use of beam search. Further, there is no decrease in uniqueness of the output molecules 170 in instances where #Uni(M₁)<N and #Uni(M_{K})≥N are true, or where #Uni(M₁)<N and #Uni(M_{K})<N are true.

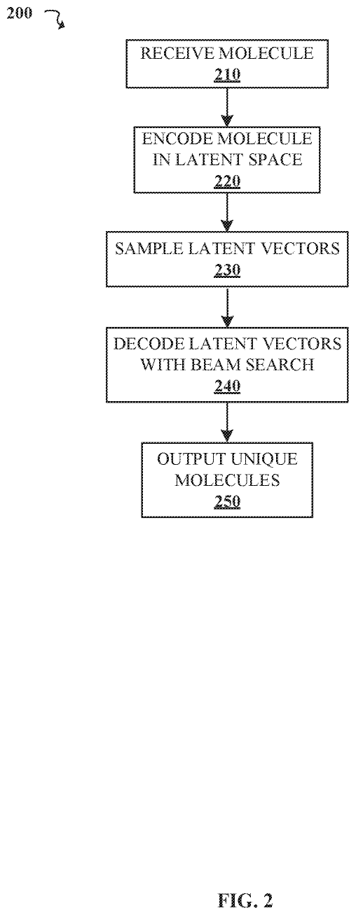

FIG. 2 is a flow diagram illustrating a process 200 of molecular design, according to some embodiments of the present disclosure. To illustrate process 200, but not to limit embodiments, FIG. 2 is described within the context of the molecular design environment 100 of FIG. 1. Where elements referred to in FIG. 2 are identical to elements shown in FIG. 1, the same reference numbers are used in both Figures.

A molecule 130 is received in a machine-readable format. This is illustrated at operation 210. For example, a selected molecule 130 (e.g., a known drug, catalyst, resin, etc.) can be entered as a SMILES character sequence, and received by the encoder 110. Additional examples of formats that can be used for machine-readable representations of molecules in discrete space are discussed in greater detail above. At least one constraint to be satisfied when generating new molecules can also be received at this step. For example, desired constraints on properties can be selected by a user (e.g., solubility, electrical conductivity, reactivity, etc.). In some embodiments, maximum and/or minimum values for various parameters (e.g., QED values, SA values, pH, molecular weight, etc.) can be entered.

The molecule 130 is encoded in latent space 105. This is illustrated at operation 220. The encoder 110 maps the received discrete representation of the molecule 130 to a continuous representation in latent space 105. The encoder 110 can be part of a VAE trained on data associating molecular structures with molecular properties. Training data can come from at least one source such as molecular structure databases, other chemical information databases, structure and property data extracted from publicly available documents, etc. The continuous representation is a latent vector, upon which latent space operations can be carried out. The operations can generate latent vectors representing new molecular structures.

The decoder 120 conditionally samples molecular representations (vectors) in the latent space 105. This is illustrated at operation 230. The conditional sampling can be carried out using sampling techniques, such as ABC. This is discussed in greater detail with respect to FIG. 1. The sampled representations are latent vectors from a region of the latent space 105 predicted to satisfy a given set of constraints (e.g., a set of constraints selected at operation 210). This target region is identified by the sampling module 140 based on properties associated with latent vectors in the region. The sampling module 140 can use the predictor 150, which can be a machine-learning model trained to associate properties with latent representations of molecular structures or a simulator designed to predict properties based on discrete representations of molecular structures, to identify the target region (e.g., based on a probability distribution on the latent space 105).

The decoder 120 then decodes the sampled latent vectors via a sequential decision-making process with beam search. This is illustrated at operation 240. The decoder 120 converts the sampled latent vectors into molecules in the machine-readable format used to represent the input molecule 130. Molecules are composed by sequentially building sets of symbols. Symbols are selected at each step based on their probabilities of correctly representing molecular features encoded by the latent vectors. The number of symbols selected at each step corresponds to a beam search width (K) specified by the beam search module 160. When the decoder 120 uses beam search to decode a latent vector, K is a number greater than 1 (e.g., K=3, K=10, etc.).

The decoder 120 then maps each latent vector to a unique decoded molecule. This is illustrated at operation 250. The output unique molecules 170 are selected from the K most likely sequences decoded for each latent vector at operation 240. The output molecules 170 can also be filtered based on novelty and validity. The output molecules 170 are represented by molecular structures in the same machine-readable format as the input molecule 130. For example, an input molecule 130 can be represented by a SMILES character sequence. An autoencoder trained on SMILES training data can then be used to generate new molecules 170 represented by SMILES character sequences in process 200.

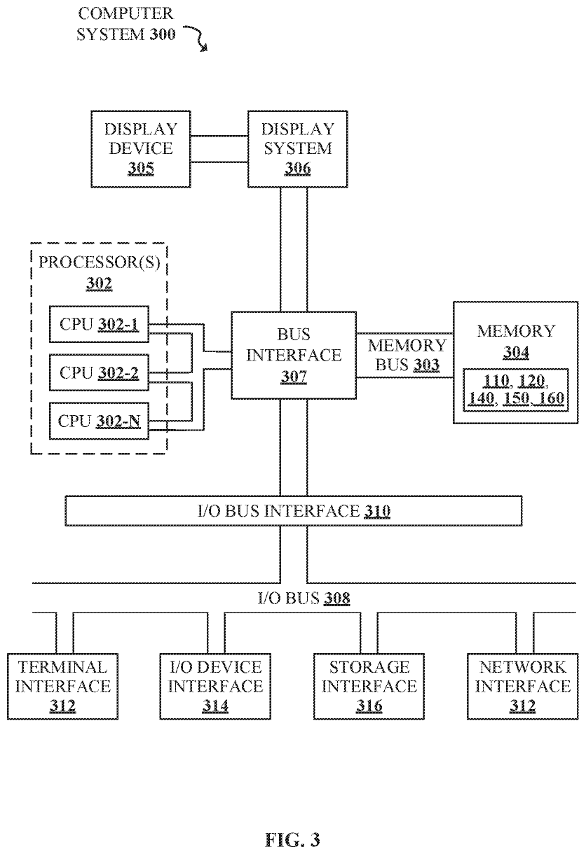

FIG. 3 is a block diagram illustrating an exemplary computer system 300 that can be used in implementing one or more of the methods, tools, components, and any related functions described herein (e.g., using one or more processor circuits or computer processors of the computer). In some embodiments, the major components of the computer system 300 comprise one or more processors 302, a memory subsystem 304, a terminal interface 312, a storage interface 316, an input/output device interface 314, and a network interface 318, all of which can be communicatively coupled, directly or indirectly, for inter-component communication via a memory bus 303, an input/output bus 308, bus interface unit 307, and an input/output bus interface unit 310.

The computer system 300 contains one or more general-purpose programmable central processing units (CPUs) 302-1, 302-2, and 302-N, herein collectively referred to as the CPU 302. In some embodiments, the computer system 300 contains multiple processors typical of a relatively large system; however, in other embodiments the computer system 300 can alternatively be a single CPU system. Each CPU 302 may execute instructions stored in the memory subsystem 304 and can include one or more levels of on-board cache.

The memory 304 can include a random-access semiconductor memory, storage device, or storage medium (either volatile or non-volatile) for storing or encoding data and programs. In some embodiments, the memory 304 represents the entire virtual memory of the computer system 300, and may also include the virtual memory of other computer systems coupled to the computer system 300 or connected via a network. The memory 304 is conceptually a single monolithic entity, but in other embodiments the memory 304 is a more complex arrangement, such as a hierarchy of caches and other memory devices. For example, memory may exist in multiple levels of caches, and these caches may be further divided by function, so that one cache holds instructions while another holds non-instruction data, which is used by the processor or processors. Memory can be further distributed and associated with different CPUs or sets of CPUs, as is known in any of various so-called non-uniform memory access (NUMA) computer architectures.

These components are illustrated as being included within the memory 304 in the computer system 300. However, in other embodiments, some or all of these components may be on different computer systems and may be accessed remotely, e.g., via a network. The computer system 300 may use virtual addressing mechanisms that allow the programs of the computer system 300 to behave as if they only have access to a large, single storage entity instead of access to multiple, smaller storage entities. Thus, though the encoder 110, decoder 120, sampling module 140, predictor 150, and beam search module 160 (FIG. 1) are illustrated as being included within the memory 304, components of the memory 304 are not necessarily all completely contained in the same storage device at the same time. Further, although these components are illustrated as being separate entities, in other embodiments some of these components, portions of some of these components, or all of these components may be packaged together.

In an embodiment, the encoder 110, decoder 120, sampling module 140, predictor 150, and beam search module 160 include instructions that execute on the processor 302 or instructions that are interpreted by instructions that execute on the processor 302 to carry out the functions as further described in this disclosure. In another embodiment, the encoder 110, decoder 120, sampling module 140, predictor 150, and beam search module 160 are implemented in hardware via semiconductor devices, chips, logical gates, circuits, circuit cards, and/or other physical hardware devices in lieu of, or in addition to, a processor-based system. In another embodiment, the encoder 110, decoder 120, sampling module 140, predictor 150, and beam search module 160 include data in addition to instructions.

Although the memory bus 303 is shown in FIG. 3 as a single bus structure providing a direct communication path among the CPUs 302, the memory subsystem 304, the display system 306, the bus interface 307, and the input/output bus interface 310, the memory bus 303 can, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the input/output bus interface 310 and the input/output bus 308 are shown as single respective units, the computer system 300 may, in some embodiments, contain multiple input/output bus interface units 310, multiple input/output buses 308, or both. Further, while multiple input/output interface units are shown, which separate the input/output bus 308 from various communications paths running to the various input/output devices, in other embodiments some or all of the input/output devices may be connected directly to one or more system input/output buses.

The computer system 300 may include a bus interface unit 307 to handle communications among the processor 302, the memory 304, a display system 306, and the input/output bus interface unit 310. The input/output bus interface unit 310 may be coupled with the input/output bus 308 for transferring data to and from the various input/output units. The input/output bus interface unit 310 communicates with multiple input/output interface units 312, 314, 316, and 318, which are also known as input/output processors (IOPs) or input/output adapters (IOAs), through the input/output bus 308. The display system 306 may include a display controller. The display controller may provide visual, audio, or both types of data to a display device 305. The display system 306 may be coupled with a display device 305, such as a standalone display screen, computer monitor, television, or a tablet or handheld device display. In alternate embodiments, one or more of the functions provided by the display system 306 may be on board a processor 302 integrated circuit. In addition, one or more of the functions provided by the bus interface unit 307 may be on board a processor 302 integrated circuit.

In some embodiments, the computer system 300 is a multi-user mainframe computer system, a single-user system, or a server computer or similar device that has little or no direct user interface, but receives requests from other computer systems (clients). Further, in some embodiments, the computer system 300 is implemented as a desktop computer, portable computer, laptop or notebook computer, tablet computer, pocket computer, telephone, smart phone, network switches or routers, or any other appropriate type of electronic device.

It is noted that FIG. 3 is intended to depict the representative major components of an exemplary computer system 300. In some embodiments, however, individual components may have greater or lesser complexity than as represented in FIG. 3, Components other than or in addition to those shown in FIG. 3 may be present, and the number, type, and configuration of such components may vary.

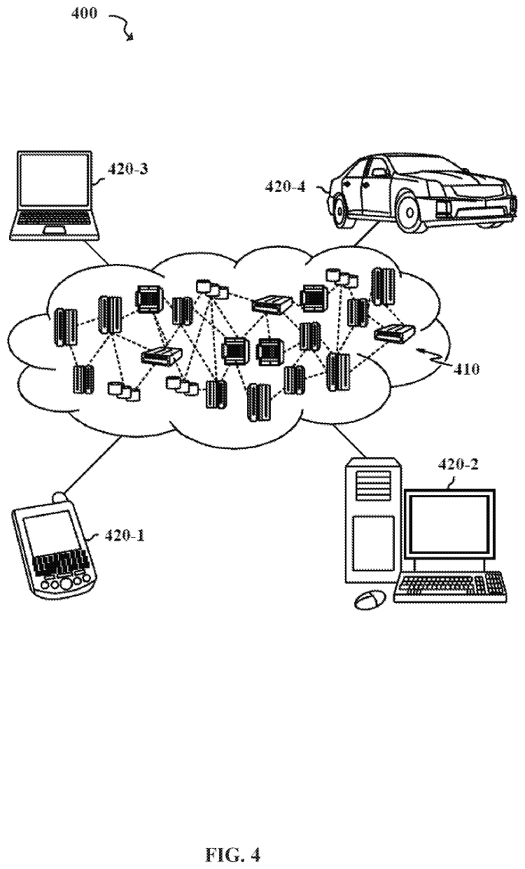

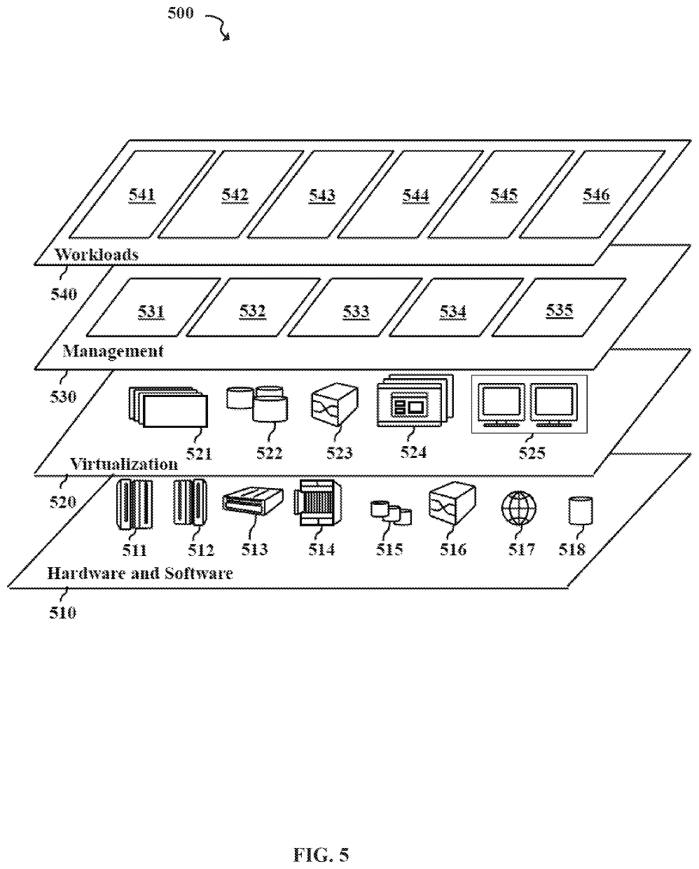

In some embodiments, the data storage and retrieval processes described herein could be implemented in a cloud computing environment, which is described below with respect to FIGS. 4 and 5. It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher-level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

FIG. 4 is a block diagram illustrating a cloud computing environment 400, according to some embodiments of the present disclosure. As shown, cloud computing environment 400 includes one or more cloud computing nodes 410 with which local computing devices used by cloud consumers, such as, for example, personal digital assistant (PDA) or cellular telephone 420-1, desktop computer 420-2, laptop computer 420-3, and/or automobile computer system 420-4 may communicate. Nodes 410 may communicate with one another. They may be grouped (not shown) physically or virtually, in one or more networks, such as Private, Community, Public, or Hybrid clouds as described hereinabove, or a combination thereof. This allows cloud computing environment 400 to offer infrastructure, platforms and/or software as services for which a cloud consumer does not need to maintain resources on a local computing device. It is understood that the types of computing devices 420-1-420-4 shown in FIG. 4 are intended to be illustrative only and that computing nodes 410 and cloud computing environment 400 can communicate with any type of computerized device over any type of network and/or network addressable connection (e.g., using a web browser).

FIG. 5 is a block diagram illustrating a set of functional abstraction model layers 500 provided by the cloud computing environment 400, according to some embodiments of the present disclosure. It should be understood in advance that the components, layers, and functions shown in FIG. 5 are intended to be illustrative only and embodiments of the invention are not limited thereto. As depicted, the following layers and corresponding functions are provided:

Hardware and software layer 510 includes hardware and software components. Examples of hardware components include: mainframes 511; RISC (Reduced Instruction Set Computer) architecture-based servers 512; servers 513; blade servers 514; storage devices 515; and networks and networking components 516. In some embodiments, software components include network application server software 517 and database software 518.

Virtualization layer 520 provides an abstraction layer from which the following examples of virtual entities may be provided: virtual servers 521; virtual storage 522; virtual networks 523, including virtual private networks; virtual applications and operating systems 524; and virtual clients 525.

In one example, management layer 530 provides the functions described below. Resource provisioning 531 provides dynamic procurement of computing resources and other resources that are utilized to perform tasks within the cloud computing environment. Metering and Pricing 532 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 533 provides access to the cloud computing environment for consumers and system administrators. Service level management 534 provides cloud computing resource allocation and management such that required service levels are met. Service Level Agreement (SLA) planning and fulfillment 535 provide pre-arrangement for, and procurement of, cloud computing resources for which a future requirement is anticipated in accordance with an SLA.

Workloads layer 540 provides examples of functionality for which the cloud computing environment can be utilized. Examples of workloads and functions that can be provided from this layer include: mapping and navigation 541; software development and lifecycle management 542; virtual classroom education delivery 543; data analytics processing 544; transaction processing 545; and computational molecular design 546.

The present disclosure may be a system, a method, and/or a computer program product. The computer program product may include a computer readable storage medium (or media) having computer readable program instructions thereon for causing a processor to carry out aspects of the present disclosure.

The computer readable storage medium is a tangible device that can retain and store instructions for use by an instruction execution device. Examples of computer readable storage media can include an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a component, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the present disclosure.

What is claimed is:

1. A system for computational molecular design, comprising:
at least one processing component;
at least one memory component;
an encoder configured to:
receive an input molecule; and
encode the input molecule as a vector in latent space;
a sampling module configured to:
identify a target region in the latent space; and
a decoder configured to:
sample latent vectors from the target region;
generate two or more discrete representations of molecules for each of the sampled latent vectors by decoding the sampled latent vectors via sequential decision-making with a beam search module configured to select a number of most likely symbols at each step in the sequential decision-making; and
output, for each of the sampled latent vectors, a unique molecule selected from the two or more discrete representations of the molecules.

2. The system of claim 1, further comprising a predictor configured to predict molecular properties based latent space representations of molecules.

3. The system of claim 1, further comprising a predictor configured to predict molecular properties based on discrete representations of molecules.

4. The system of claim 1, wherein the sampling module identifies the target region based on a probability distribution on the latent space.

5. The system of claim 1, wherein the number of the most likely symbols is greater than 1.

6. The system of claim 1, wherein the most likely symbols have the highest probabilities of satisfying a given constraint.

7. The system of claim 5, wherein the given constraint is a threshold quantitative estimate of drug likeness value.

8. The system of claim 1, wherein the encoder and the decoder are based on a recurrent neural network.

9. A method of computational molecular design, comprising:
receiving an input molecule;
encoding the input molecule as a vector in latent space;
identifying a target region in the latent space;
sampling latent vectors from the target region;
generating two or more discrete representations of molecules for each of the sampled latent vectors by decoding the sampled latent vectors via sequential decision-making, wherein the sequential decision-making includes selecting a number of most likely symbols at each step; and
outputting, for each of the sampled latent vectors, a unique molecule selected from the two or more discrete representations of molecules.

10. The method of claim 9, further comprising predicting molecular properties based on latent space representations of molecules.

11. The method of claim 9, further comprising predicting molecular properties based on the discrete representations of molecules.

12. The method of claim 9, wherein the target region is identified based on a probability distribution on the latent space.

13. The method of claim 9, wherein the number of the most likely symbols is greater than 1.

14. The method of claim 9, wherein the most likely symbols have the highest probabilities of satisfying a given constraint.

15. The method of claim 9, wherein the encoding and the decoding are carried out with a recurrent neural network.

16. A computer program product for computational molecular design, the computer program product comprising a computer readable storage medium having program instructions embodied therewith, the program instructions executable by a processor to cause a device to perform a method, the method comprising:
receiving an input molecule;
encoding the input molecule as a vector in latent space;
identifying a target region in the latent space;
sampling latent vectors from the target region;
generating two or more discrete representations of molecules for each of the sampled latent vectors by decoding the sampled latent vectors via sequential decision-making, wherein the sequential decision-making includes selecting a number of most likely symbols at each step; and
outputting, for each of the sampled latent vectors, a unique molecule selected from the two or more discrete representations of molecules.

17. The computer program product of claim 16, wherein the target region is identified based on a probability distribution on the latent space.

18. The computer program product of claim 16, wherein the number of the most likely symbols is greater than 1.

19. The computer program product of claim 16, wherein the most likely symbols have the highest probabilities of satisfying a given constraint.

20. The computer program product of claim 16, wherein the encoding and the decoding are carried out with a recurrent neural network.
